(19) United States
(12) Patent Application Publication
(10) Pub. No.: US 2005/0186141 A1
(43) Pub. Date: Aug. 25, 2005

(54) Transdermal aerosol compositions

(75) Inventors: Igor Gonda, South Yarra (AU); Timothy Matthias Morgan, Carlton North (AU); Nina Frances Wilkins, Kensington (AU)

Correspondence Address: FOLEY AND LARDNER;SUITE 500, 3000 K STREET NW, WASHINGTON, DC, 20007, US

(73) Assignee: Acrux DDS Pty Ltd.

(21) Appl. No.: 11/019,121

(22) Filed: Dec. 22, 2004

Related U.S. Application Data
(63) Continuation of application No. PCT/AU03/00784, filed on Jun. 24, 2003.

(30) Foreign Application Priority Data
Jun. 25, 2002 (AU) PS3171

(51) Int. Cl. A61L 9/04; A61K 7/42; A61K 9/70

(52) U.S. Cl. 424/45; 424/59

(57) ABSTRACT

The present invention provides a pharmaceutical composition for transdermal delivery comprising: one or more physiologically active agents; one or more dermal penetration enhancers; a pharmaceutically acceptable carrier comprising a volatile solvent; and a hydrofluorocarbon propellent; wherein the carrier and penetration enhancers combine to provide a single-phase solution of the one or more physiologically active agents.

### FIELD OF THE INVENTION

The present invention relates to transdermal aerosol compositions for topical application, a spray device for transdermal delivery of aerosol compositions and to a method of transdermal delivery of therapeutic agents.

### BACKGROUND OF THE INVENTION

Conventional means for administering therapeutic agents (‘active agents’) to a human or animal are usually limited to some degree by biological, chemical, and physical barriers. Examples of physical barriers are the skin and various organ membranes that must be traversed before the agent reaches a target. Chemical barriers include pH variations, lipid bi-layers, and degrading enzymes. Both biologically and chemically active agents are particularly vulnerable to such barriers.

Transdermal delivery of therapeutic agents offers several inherent clinical and patient advantages over traditional oral tablet and capsule formulations, especially for drugs that:
cannot safely be given orally, for example because of irritant effects on the gastrointestinal tract
undergo extensive so-called ‘first-pass’ metabolism and are thus substantially inactivated in the liver immediately after oral administration
are poorly absorbed or poorly bioavailable after oral administration
are best delivered in small, consistent quantities over a long period, rather than in ‘spikes’, which may be associated with side-effects.

Administration of physiologically active agents through the skin (‘transdermal drug delivery’) has received increased attention because it not only provides a relatively simple dosage regime but it also provides a relatively slow and controlled route for release of a physiologically active agent into the systemic circulation. However, transdermal drug delivery is complicated by the fact that the skin behaves as a natural barrier and therefore transport of agents through the skin is a complex mechanism.

Structurally, the skin consists of two principle parts, a relatively thin outermost layer (the ‘epidermis’) and a thicker inner region (the ‘dermis’). The outermost layer of the epidermis (the ‘stratum corneum’) consists of flattened dead cells which are filled with keratin. The region between the flattened dead cells of the stratum corneum is filled with lipids which form lamellar phases that are responsible for the natural barrier properties of the skin.

For effective transdermal delivery of a physiologically active agent that is applied to the surface of the skin (‘topical application’), the agent must be partitioned firstly from the vehicle into the stratum corneum, it must typically then be diffused within the stratum corneum before being partitioned from the stratum corneum to the viable epidermis, dermis and into the bloodstream.

To overcome some of the problems with transdermal delivery that are associated with transport across the dermal layers (‘percutaneous absorption’), physiologically active agents can be formulated with incorporation of one or more drug penetration enhancers. For example, aqueous ethanol can be used as a vehicle in formulations for topical application. Ethanol can act as a penetration enhancer that can increase the flux of an active agent across the skin due to a solvent drag effect (Berner et al., 1989, J. Pharm. Sci, 78(5), 402-406). Octyl para-methoxycinnamate (Padimate O), Octyl salicylate and Azone™ are further examples of penetration enhancers that have been shown to improve percutaneous absorption (U.S. Pat. No. 6,299,900).

PCT/AU00/01419 describes a propellant free spray on skin patch composition, which forms a flexible porous skin patch to improve wound healing and drug administration, however the composition is limited to water soluble compounds.

The use of a transdermal aerosol drug delivery system has the potential to overcome the limitations of existing transdermal drug delivery devices, such as transdermal patches. In particular, the potential to avoid skin irritation (Morgan et al., 1998, J. Pharm. Sci. 87, 1226-28) offers a significant advantage over existing patch and nasal delivery methods, both of which have been shown to cause application site reactions in up to 50% of patients using these types of dosage forms (Lopes et al., 2001, Maturitas 38, S31-39).

U.S. Pat. No. 6,325,990 relates to a film forming composition for spraying on the skin comprising a physiological active, a polysiloxane adhesive, an absorption promoter, a solvent, a volatile silicone and a propellant. We have found that this invention suffers from a number of disadvantages.

In transdermal systems where both a drug and an enhancer are incorporated, it is important that the enhancer is released at a rate that will result in an optimal effect upon drug permeation through the skin. Therefore, in a film-forming system, the adhesive must show effective permeability for the drug and the enhancer, defined by the delivery profile of the drug under consideration. If the solubility of either the drug or the enhancer is not optimised, then the permeation profile will be affected (Venkatraman et al., 1998). Drug-in-adhesive systems are more recent second generation systems wherein the drug is dispersed in the adhesive itself. The saturated solubility for many compounds in adhesives is low, thus the tendency for the drug to precipitate is even greater, leading to stability issues. (Kotiyan et al., 2001).

Liquid excipients (including the drug) will ‘plasticise’ the adhesive to some degree; which would lead to an undesirable residue on the skin. This “plasticised” residue is often sticky, collecting dirt and lint, and is therefore cosmetically unacceptable.

There is a need for an effective transdermal composition which can be easily applied to the skin and which provides effective transdermal administration.

Not surprisingly, it has been found that to date there is no metered dose transdermal aerosol composition that improves percutaneous delivery by the appropriate selection propellant and solvent, existing as a single-phase solution, with a penetration enhancer of choice and without leaving a residue or film.

No admission is made that any reference, including any patent or patent document, cited in this specification constitutes prior art. In particular, it will be understood that, unless otherwise stated, reference to any document herein does not constitute an admission that any of these documents forms part of the common general knowledge in the art in Australia or in any other country. The discussion of the references states what their authors assert, and the applicant reserves the right to challenge the accuracy and pertinency of any of the documents cited herein.

### SUMMARY OF THE INVENTION

The present invention arises from the inventor's studies of finite dose formulations which contain penetration enhancers that enhance the percutaneous absorption of a therapeutic agent.

The present invention arises, at least in part, from the realisation that an improvement in percutaneous delivery can be achieved by the appropriate selection of a hydrofluorocarbon propellant dissolved in a lower alcohol such as ethanol or isopropyl alcohol or a combination thereof, and which can also exist as a single-phase solution with the penetration enhancer of choice. Additionally, the aerosol composition may initially contain water in an amount up to 50% w/v preferably up to 10% w/v water, and more preferably may initially contain up to 5% w/v water without impacting upon the capacity of the volatile vehicle to dissolve the desired amount of the therapeutic agent and penetration enhancer used in said metered-dose transdermal aerosol compositions in their most preferred form as single-phase solutions.

Accordingly, in a first form the present invention provides a composition including:
one or more physiologically active agents;
one or more dermal penetration enhancers; and
a volatile pharmaceutically acceptable solvent comprising a lower alcohol and a hydrofluorocarbon propellant, and optionally up to 50% w/v water wherein the physiologically active agent, dermal penetration enhancer, volatile pharmaceutically acceptable solvent and propellant combine to preferably form a single-phase solution.

Compositions with a relatively higher water content of up to 50% w/v water may be used in a topical vehicle that can be applied to irritated skin, broken skin or mucous membranes, wherein the composition may exist as a single phase solution, emulsion or micro-emulsion in which the active agent and/or penetration are either completely dissolved within one of the aforementioned vehicle phases or are alternatively dispersed within one of these vehicle phases, or a combination thereof, such as the physiologically active agent being dissolved in the composition and the dermal penetration enhancer being dispersed in the same composition.

Compositions comprising water in an amount of up to 10% w/v are preferred.

The composition of the present invention may overcome at least some of the disadvantages of the composition described in the aforementioned U.S. Pat. No. 6,325,990, which can result in a two phase solution or emulsion, as opposed to the single phase solution of the present invention.

Water uptake in polysiloxane systems such as the one described in U.S. Pat. No. 6,325,990 is a challenging issue due to the irreversible changes to the polymer properties that water brings about. For example, it has been shown that entrance of water induces both a swelling of the system and a break in the adhesive bonding capability (Cabanelas, et al., 2003). Any absorption of water during storage of compositions such as the one described in U.S. Pat. No. 6,325,990 may result in a change in the physical properties of the vehicle phase separation, leading to a decrease in the leaving tendency of the physiological active and subsequent decline in percutaneous penetration and/or a need to shake the container holding the vehicle prior to its application to the skin.

The present invention also provides a metered dose spray applicator containing the above composition for transdermal administration.

The present invention further provides a method of treatment of a subject with a physiologically active agent comprising applying a transdermal composition as hereinbefore described to an area of skin of a subject.

### DETAILED DESCRIPTION OF THE PREFERRED EMBODIMENT

The composition of the invention comprises a hydrofluorocarbon propellant. The hydrofluorocarbon propellant is preferably a hydrofluoroalkane such as HFC-134a or HFC 127. The most preferred hydrofluorocarbon propellant is HFC-134a.

We have found that HFC-134a is particularly advantageous in compositions to be administered transdermally as compositions of the invention applied to the skin with HFC-134a produce more saturation of the drug when compared with other propellants such as dimethyl ether. We have found that rapidly providing high saturation of the active and penetration enhancer on the skin increases partitioning of the drug and penetration enhancer into the skin rapidly providing a reservoir of active and penetration enhancer within the skin. In addition, we have found that incorporation of HFC-134a provides for a faster drying time which allows the physiological active and the penetration enhancer to form an amorphous deposit upon evaporation of the volatile carrier. Upon delivery of the composition to the skin, it is preferable that the volatile solvent evaporates and the composition becomes touch dry within 2 minutes, more preferably within 1 minute, leaving no residue or film on the skin.

The amount of propellant in the composition of the invention is preferably 15 to 50% v/v and more preferably 20 to 40% v/v.

The composition of the invention contains a penetration enhancer. The preferred penetration enhancers for use in the composition of the invention are sunscreen esters of formula (I): 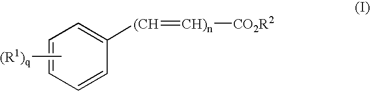
wherein
R¹ is hydrogen, lower alcohol, lower alkoxy, halide, hydroxy or NR³R⁴;
R² is a C₈ to C₁₈ alkyl,
R³ and R⁴ are each independently hydrogen, lower alkyl or R³ and R⁴ together with the nitrogen atom to which they are attached form a 5- or 6-membered heterocyclic ring;
n is 0 or 1,
q is 1 or 2,
wherein when n is 0 and R1 is NR³R⁴, the NR³N⁴ is para-substitued.

Particularly preferred sunscreen esters are those selected from the group consisting of C₈ to C₁₈ alkylcinnamate, C₈ to C₁₈ alkylmethoxycinnamate, C₈ to C₁₈ alkyl salicylate and mixtures thereof. More preferably the penetration enhancers are selected from padimate O and octyl salicylate.

The amount of penetration enhancer present in the composition of the invention is preferably in the range of 0.1 to 10% w/v and more preferably 2 to 8% w/v.

The composition of the invention contains a lower alcohol, preferably ethanol, propanol (including isomers thereof) or a mixture thereof. Preferably the volatile solvent comprises at least 0.60% w/v of one or more lower alcohols. More preferably the volatile solvent component consists essentially of an ethanol, isopropanol or mixture thereof. It is present in an amount sufficient to provide a single phase with the penetration enhancer and propellant. Typically the alcohol will be present in an amount of from 40 to 80% v/v and more preferably 50 to 70% v/v.

The choice of solvent used in a composition can be selected on the basis of the desired transdermal delivery profile as measured by percutaneous penetration in order to achieve the desired pharmacological effect. Combinations of volatile solvents could be used to obtain the desired pharmacological effect; for example on a weight basis:

| | |
|---|---|
| Ethanol: Isopropyl Alcohol (IPA) | 20-80:20-80 |
| Ethanol or IPA: Acetone or Chloroform | 60-90:10-40; |

or a mixture thereof.

The composition of the invention may contain water. The decision on whether water is to be present and the amount of water will depend on the active physiological agent and its stability and interaction with water and whether the composition is to be applied to irritated skin, broken skin or mucous membranes. In some instances water may be a useful solvent whereas in other circumstances instability of the active in the presence of water may dictate that water be omitted. Indeed in some cases special precautions against the presence of water such as the use of desiccants may be desirable.

The composition of the invention includes a physiologically active agent. Examples of suitable physiologically active agents include steroid, hormone derivative, non-steroidal anti-inflammatory drug, opioid analgesic, antinauseant, antioestrogen, aromatase inhibitor, 5-alpha reductase inhibitor, anxiolytic, prostaglandin, anti-viral drug, anti-migraine compound, antihypertensive agent, anti-malarial compound, bronchodilator, anti-depressant, anti-alzheimer's agent, neuroleptic and antipsychotic agent, anticholinergic agent, anti-parkinson's agent antiandrogen or anorectic agent.

The preferred physiologically acceptable agents include testosterone, oestradiol, ethinyloestradiol, levonorgestrel, progesterone, norethisterone acetate, ibuprofen, ketoprofen, flurbiprofen, naproxen, diclofenac, fentanyl, buprenorphine, scopolamine, prochlorperazine, metochlopramide, ondansetron, tamoxifen, epitiostanol, exemestane, oxybutynin, darifenacin, tolterodine, ropinirole, granisetron, rivastigmine, buspirone, rizatriptin, zolmitriptan, lacidipine, tropisetron, olanzapine and methyl phenidate, 4-hydroxyandrostenedione and its derivatives, finasteride, dutasteride, turosteride, LY191704, MK-386, alprazolam, alprostadil, prostacylcin and its derivatives, melatonin, n-docosanol, tromantadine, lipophilic pro-drugs of acyclovir, low molecular weight heparin, enoxaparin, sumatriptan, amlodipine, nitrendipine, prim aquine, minoxidi, minoxidil pro-drugs, pilocarpine, salbutamol, terbutaline, salmeterol, ibogaine, bupropian, rolipram, tacrine, fluphenazine, haloperidol, N-0923, cyproterone acetate, MENT (7-methyl-19-testosterone), or mazindol or an pharmaceutically acceptable salt or derivative of any one of the aforementioned.

Examples of suitable anticholinergic agents include oxybutynin, darifenacin and tolterodine.

More preferably the physiologically acceptable agents include apomorphine, oxybutynin, ropinirole, fentanyl, granisetron, rivastigmine, buspirone, rizatriptin, zolmitriptan, lacidipine, tropisetron, olanzapine and methyl phenidate or a pharmaceutically acceptable salt or derivative of any one of the aforementioned.

One aspect of the invention provides a metered dose spray applicator containing a composition for transdermal administration. The composition of the invention will generally be retained under pressure within the container so that a significant proportion of the propellant is in liquid form. The spray applicator may comprise a nozzle and means for providing a metered dose of spray from the nozzle. The spray applicator may further comprise spacing means for spacing the application nozzle at a predetermined distance from the skin of the subject onto which the spray is to be delivered.

The invention will now be described with reference to the following examples. It is to be understood that the examples are provided by way of illustration of the invention and that they are in no way limiting to the scope of the invention.

### EXAMPLE 1

An aerosol composition for transdermal delivery of an analgesic was prepared from the following composition.

| | |
|---|---|
| Fentanyl | 5% w/v |
| Octyl salicylate | 8% w/v |
| HFC-134a | 30% v/v |
| IPA (95%) | to volume |

### EXAMPLE 2

An aerosol composition for transdermal delivery of a non-steroidal anti-inflammatory drug was prepared as a single phase solution, using the following components:

| | |
|---|---|
| Ketoprofen | 5% w/v |
| Octyl salicylate | 4% w/v |
| HFC-134a | 30% v/v |
| Ethanol (95%) | to volume |

### EXAMPLE 3

An aerosol composition for transdermal delivery of an anti-cholinergic drug was prepared as a single phase solution from the composition described below.

| | |
|---|---|
| Oxybutynin | 5% w/v |
| Octyl salicylate | 2.5% w/v |
| HFC-134a | 30% v/v |
| IPA (95%) | to volume |

### EXAMPLE 4

An aerosol composition for transdermal delivery of an anti-anxiety drug to the skin was prepared as a single phase solution from the following composition:

| | |
|---|---|
| Buspirone | 4% w/v |
| Octyl salicylate | 5% w/v |
| HFC-134a | 30% v/v |
| Ethanol (95%) | to volume |

### EXAMPLE 5

An aerosol composition for transdermal delivery of an anti-Parkinson agent was prepared as a single phase solution from the composition described below.

| | |
|---|---|
| Ropinirole | 5% w/v |
| Octyl salicylate | 5% w/v |
| HFC-134a | 30% v/v |
| IPA (95%) | to volume |

### EXAMPLE 6

| | |
|---|---|
| Granisetron | 5% w/v |
| Octyl salicylate | 8% w/v |
| HFC-134a | 30% v/v |
| Ethanol (95%) | to volume |

### EXAMPLE 7

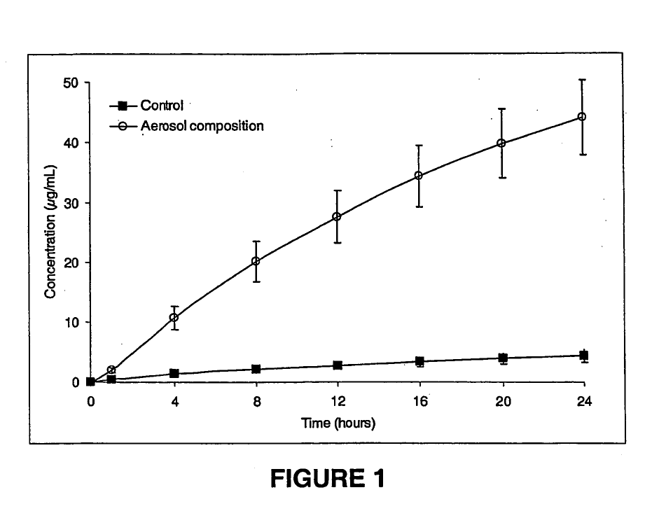

Example 7 is described with reference to the attached drawing. In the drawing FIG. 1 is a graph showing skin penetration of buspirone over time.

The use of an HFC propellant in a composition will produce a single phase solution with better drug saturation when compared with other propellants. By providing high saturation of the active and penetration enhancer on the skin, a amorphous deposit of drug within the stratum corneum can be achieved. As a result an increase in the penetration of a drug across the skin can be expected as shown in FIG. 1.

Finally, it is to be understood that various other modifications and/or alterations may be made without departing from the spirit of the present invention as outlined herein.

1. A pharmaceutical composition for transdermal delivery comprising;
one or more physiologically active agents;
one or more dermal penetration enhancers;
a pharmaceutically acceptable carrier comprising a volatile solvent; and
a propellent comprising HFC-134a;
wherein the carrier, propellant and penetration enhancer are combined to provide a single-phase solution of the one or more physiologically active agents.

2. A pharmaceutical composition according to claim 1 wherein the volatile solvent has a vapour pressure above 35 mmHg at atmospheric pressure and a temperature of 32° C.

3. A composition according to claim 1 substantially free of adhesives for forming a film on the skin.

4. A composition according to claim 1 wherein said composition maintains a drying time of less than 2 minutes, more preferably less than 1 minute.

5. (canceled)

6. A composition according to claim 4 wherein said propellent consists essentially of HFC-134a.

7. A pharmaceutical composition according to claim 1 wherein the propellant is from 15% to 50% by volume of the total pharmaceutical composition.

8. A pharmaceutical composition according to claim 7 wherein the propellant is from 20 to 40% by volume of the composition.

9. A pharmaceutical composition according to claim 1 wherein the penetration enhancer comprises one or more sunscreen esters.

10. (canceled)

11. A pharmaceutical composition according to claim 9 wherein the one or more sunscreen esters is selected from the group consisting of C₈ to C₁₈ alkylcinnamate, C₈ to C₁₈ alkylmethoxycinnamate, C₈ to C₁₈ alkyl salicylate and mixtures thereof.

12. A pharmaceutical composition according to claim 11 wherein the penetration enhancer is octyl salicylate or padimate-o.

13. (canceled)

14. A pharmaceutical composition according to claim 9 wherein the composition comprises from 0.1% to 10% by weight of dermal penetration enhancer.

15. (canceled)

16. (canceled)

17. (canceled)

18. A pharmaceutical composition according to claim 1 wherein the solvent comprises ethanol, isopropanol or a mixture thereof, providing a single phase of penetration enhancer and propellent.

19. A pharmaceutical composition according to claim 1 wherein the volatile solvent comprises acetone, chloroform, lower alcohol or mixtures thereof and is present in from 40% to 80% by volume of the total pharmaceutical composition.

20. A pharmaceutical composition according to claim 1 wherein the volatile solvent comprises acetone, chloroform, a lower alcohol or mixtures thereof and is present in from 50% to 70% by volume of the total pharmaceutical composition.

21. A pharmaceutical composition according to claim 1 comprising one or more physiologically active agents selected from the group consisting of steroid, hormone derivative, non-steroidal anti-inflammatory drug, opioid analgesic, antinauseant, antioestrogen, aromatase inhibitor, 5-alpha reductase inhibitor, anxiolytic, prostaglandin, anti-viral drug, anti-migraine compound, antihypertensive agent, anti-malarial compound, bronchodilator anti-depressant, anti-Alzheimer's agent, anticholinergic agent, neuroleptic and antipsychotic agent, anti-Parkinson's agent, antiandrogen and anoretic agent.

22. A pharmaceutical composition according to claim 21 wherein the one or more physiologically acceptable agents is selected from the group consisting of testosterone, oestradiol, ethinyloestradiol, levonorgestrel, progesterone, norethisterone acetate, ibuprofen, ketoprofen, flurbiprofen, naproxen, diclofenac, fentanyl, buprenorphjne, scopolamine, prochlorperazine, metochlopramide, ondansetron, tamoxifen, epitiostanol, exemestane, darifenacin, 4-hydroxyandrostenedione and it's derivatives, finasteride, dutasteride, turosteride, LY191704, MK-386, alprazolam, alprostadil, prostacyclin and it's derivatives, melatonin, n-docosanol, tromantadine, lipophilic pro-drugs of acyclovir, low molecular weight heparin, enoxaparin, sumatriptan, amlodipine, nitrendipine, primaquine, minoxidiland it's pro-drugs, pilocarpine, salbutamol, terbutaline, sameterol, ibogaine, bupropian, rolipram, tacrine, fluphenazine, haloperidol, N-0923, cyproterone acetate, MENT (7-methyl-19-testosterone), or mazindol or a pharmaceutically acceptable salt or derivative of any one of the aforementioned. More preferably, the physiological agents include apomorphine, oxybutynin, fentanyl, ropinirole, granisetron, rivastigmine, buspirone, rizatriptin, zolmitriptan, lacidipine, tropisetron, olanzapine and methyl phenidate or a pharmaceutically acceptable salt or derivative of any one of the aforementioned.

23. A pharmaceutical composition according to claim 1 wherein the composition is contained in a chamber of a spray applicator device comprising a valve for delivering the composition from the chamber, a nozzle for dispersing the composition as an aerosol and means for providing a metered dose of aerosol from the nozzle said composition being retained under pressure within the chamber so as to maintain said propellent in a liquid form.

24. (canceled)

25. A method of transdermal delivery of a physiologically active agent to a subject forming a composition of the physiologically active agent comprising a dermal penetration enhancer and pharmaceutically acceptable carrier comprising a volatile solvent and HFC-134a propellant to provide a single phase solution under pressure; applying the composition as an aerosol to the skin of the subject.
